(19) United States
(12) Patent Application Publication
(10) Pub. No.: US 2023/0399689 A1
(43) Pub. Date: Dec. 14, 2023

(54) SYSTEMS AND METHODS FOR INTEGRATION OF MICROBIAL SEQUENCING WITH GROWTH RECIPES

(71) Applicant: BATTELLE MEMORIAL INSTITUTE, Columbus, OH (US)

(72) Inventor: David C. Glasbrenner, Columbus, OH (US)

(73) Assignee: Battelle Memorial Institute, Columbus, OH (US)

(21) Appl. No.: 18/208,679

(22) Filed: Jun. 12, 2023

Related U.S. Application Data
(60) Provisional application No. 63/351,338, filed on Jun. 10, 2022.

(51) Int. Cl. C12Q 1/6869; G16B 30/00; C12Q 1/6888

(52) U.S. Cl. CPC C12Q 1/6869; G16B 30/00; C12Q 1/6888

(57) ABSTRACT

The invention relates to microbial growth. The invention provides a system and method for rapidly and reliably generating information for microbial growth for known and unknown microorganisms. Using sequence information of the microorganisms in a sample, the system and method identifies the microorganisms contained in the sample and generates growth recipes and conditions for each. The recipes are identified from a recipe database or alternatively created by taking into consideration the species, unique characteristics and growth requirements of the user-desired microbe.

### CROSS-REFERENCE TO RELATED APPLICATIONS

This application claims priority benefit of U.S. Provisional Application Ser. No. 63/351,338, filed on Jun. 10, 2022; which is incorporated herein by reference.

### FIELD OF THE INVENTION

The present invention is directed to the detection, identification, analysis and propagation of microorganisms. In particular, a method to facilitate growth of desired microorganisms in a rapid and reliable manner.

### INTRODUCTION

The identification and rapid propagation of microorganisms is of vital importance to a wide range of fields ranging from environmental, industrial, biodefense and many areas of public health.

Disease outbreaks such as COVID-19 and derivatives have reinforced the critical need in rapid prototyping of microbes. The term “microbe” as used herein refers to any of bacteria, archaea, fungi, virus, or eukaryote. For highly infectious viral agents, bacteria, or fungi, developing countermeasures such as inoculations and vaccines, anti-bacterial or anti-fungal programs or other medical treatments, requires rapid identification of the microbe as well as production of sufficient quantities to supply laboratories possibly on a worldwide basis. Research laboratories rely on bulk quantities of microbial samples in order to analyze and advance effective therapeutic strategies to contain and treat the infection. During disease outbreaks, a delay by few days in identifying and propagating the infectious microbe, can translate statistically to the cost of disease, lost lives, and economic hardship. Additionally, the ability of microbes to constantly undergo genetic mutations and present varying phenotypes, necessitates a constant need for identifying and routinely maintaining new organisms for the purpose of research inquiry and in preparation to protect against future disease outbreaks by these microbes or those initiated by related species.

Currently, there are numerous well-established approaches to microbe identification. Typically, these approaches utilize sequence information of the microbe, derived from either nucleic acid or protein; in other cases, the morphology, physiology or biochemical tests provides sufficient information for microbe identification.

Sequencing DNA, RNA or protein for identification means determining the order and nature of chemical building blocks that appear unique to a species. Databases and computer-based tools are then leveraged to analyze the genome, transcriptome, or proteome to provide species resolution for the microbe under study. Even simple virus genomes may be astoundingly diverse with respect to size, complexity, and type of nucleic acid. Viral genomes may consist of DNA or RNA, may be double or single stranded, monopartite, or multipartite, short sequences of about 2 kilobases (˜2 kb) or long sequences 2500 kilobases (˜2500 kb).

Persons with ordinary skill in the art may use various sequence profiling tools to input actual DNA, RNA or protein sequences and automate and integrate the process of searching different DNA, RNA and protein databases to determine the organism under study. Alternatively, microscopic visualization techniques may be used to examine morphology to identify or categorize an apparently unknown microbe. Although the morphology of a microbe maybe relatively stable under suitable conditions, temporal and environmental conditions can contribute to altered states of morphology, that can complicate interpretation. Additionally, understanding morphology or physiology requires growth of the microorganisms as colonies (group of individual microbial units), requiring a prior understanding of specific growth conditions to maintain or propagate the colony.

Irrespective of whether sequence information or morphologic evaluation is used to discern the microbial species, there has been no effort to identify the most optimal growth conditions that are unique to a microbial species. Currently, if an unknown microbe needs to be cultured in the laboratory, manual processes of some kind often are required to make an educated guess as to the appropriate recipe that will optimize growth and development of the microbe. Often, these manual processes may involve search and review of technical literature and, as a result, some processes may take hours or days to complete. Moreover, current recipe choices appear to have non-optimal success rates, as approximately only 1% of known bacterial species have well-defined growth conditions, tested in a laboratory setting.

Consequently, if an incorrect recipe is used to grow a particular microbe, the microbe can die. An example of this scenario is incorrectly adding growth nutrients for a specific microbe that is recommended for growth of a different microbial species. Specifically, if a recipe for E. coli is used for Prochloroccus, the Prochloroccus would not be able to grow. Tables 1 and 2 show these two recipes as examples:

TABLE 1
Example recipe for Prochlorococcus

| | | Primary Stock | Dilution | Final Conc. |
|---|---|---|---|---|
| Nutrient | Grade | (M) | Factor | (μM) |
| NaH₂PO₄•H₂O | SigmaUltra | 0.025 | 1:500 | 50 |
| NH₄Cl | SigmaUltra | 0.50 | 1:625 | 800 |
| Na₂EDTA•2 H₂O | 99% | 0.012 | 1:10⁴ | 1.17 |
| FeCl₃•6 H₂O | Analytic | 0.012 | 1:10⁴ | 1.18 |
| ZnSO₄•7 H₂O | >99.5% | 0.080 | 1:10⁷ | 0.008 |
| CoCl₂•6 H₂O | Analytic | 0.050 | 1:10⁷ | 0.005 |
| MnCl₂•4 H₂O | Analytic | 0.900 | 1:10⁷ | 0.090 |
| Na₂MoO₄•2 H₂O | ACS | 0.030 | 1:10⁷ | 0.003 |
| Na₂SeO₃ | ~98% | 0.100 | 1:10⁷ | 0.010 |
| NiCl₂•6 H₂O | Analytic | 0.100 | 1:10⁷ | 0.010 |

TABLE 2
Example recipe for E. Coli.

| | | | |
|---|---|---|---|
| Ingredient | 1 L | 3 L | 4 L |

| | | | | | | |
|---|---|---|---|---|---|---|
| Tryptone | 10 | g | 30 | g | 40 | g |
| Yeast extract | 5 | g | 15 | g | 20 | g |
| NaCl | 10 | g | 30 | g | 40 | g |
| Water | 1 | L | 3 | L | 4 | L |

WO20200148956A1 (Matsubara et al.) describes a cell generation support device, method, and program. Culture conditions appear to be acquired depending on the history of the specific cells used as inputs to the device, rather than based on species taxa. Matsubara's system further appears to rely on numbers of input cells, and develops culture conditions accordingly. Among other aspects, the system does not appear to be integrated with a sequence for a specific microbe, nor does there appear to be user input to drive success of the growth program for future researchers.

U.S. Ser. No. 10/323,225B2 (Farmer et al.) directs to systems and methods for growing microorganisms. In Farmer, the system appears to receive a batch identification code and using that code, recovers a recipe in memory associated with the code. Like Matsubara, Farmer's system does not appear to be integrated with microbial sequencing. In addition, Farmer appears constrained to the parameter settings pre-loaded into its fermentation system. There does not appear to be an option for microbes that are not initially identified, i.e., without pre-specified batch codes.

US20160264922A1 (Ozaki et al.) describes an automatic cell management system for varying cells and cultures. Described as a “cell culture factory” Ozaki monitors the culture reports out, in real time, the kind and state of cells cultured at the time of measurement. Like Matsubara and Famer, Ozaki does not utilize identification systems, nor does it appear to develop and maintain a central recipe repository.

Despite the proliferation of diverse bioinformatics tools for genomic and proteomic genetic analysis, there is an unmet need for a system that can harness these specialized tools to create recipes for growth environments unique to known or unknown microorganisms. Using this information, the generation of optimal growth conditions for microbial propagation in a swift manner, addresses goals that align with increasing the population of beneficial microbes, retarding growth of pathogenic or harmful microbes or in providing sufficient microbes to drive the process of research and discovery.

The following disclosures improve upon the prior art by providing systems and methods for integrating microbial sequencing with recipes and conditions for microbe growth. Such a system eliminates the manual element and bottleneck of searching through literature for growth recipes, and thus microbes can be grown faster and in larger quantities. In addition, having the recipes curated into a repository and associated with a species taxa allows for better starting points to growing novel or hitherto unknown organisms, leading to improved timelines for development and culturing. Importantly, the potential of the system to generate growth conditions for desired microorganisms by evaluating a combination of microbial characteristics such as sequence, proteome, pathways, etc. allows for more accurate determinations of growth conditions.

### SUMMARY OF THE INVENTION

The present invention provides a rapid and integrated identification and propagation system for various known and unknown bacteria, other unicellular and multicellular microorganisms. The systems and methods described herein, including various embodiments, may be referred to as GrowSEQ.

A preferred embodiment of GrowSEQ relates to a method of identifying and characterizing microorganisms in a complex mixture of different microorganisms, and providing ideal growth recipes and conditions for the propagation of one or more desired species, as specified by the user.

In one aspect, GrowSEQ identifies recipes for unknown microorganisms based on relatedness to its closest identifiable species with known growth conditions.

In another aspect, GrowSEQ generates growth recipes for unknown microorganisms based on the identity and characteristics of the user-desired microorganism.

Additionally, GrowSEQ curates growth recipes from publicly available and private databases, but critically, allows user input on microbial growth and other growth metrics and leverages machine learning/artificial intelligence algorithms to learn through iterative training and challenge steps to provide increasingly accurate recipe suggestions for future unknown species.

The invention may include any of the detailed methods and/or method steps in whole or in part. It will be appreciated that in the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, systems, and components have not been described in detail so as not to unnecessarily obscure aspects of the various embodiments. In some instances, the concepts herein may obviate in whole or in part one or more of the problems encountered in the prior art.

### Glossary

As used herein, the term “microorganism”, used interchangeably with “microbe”, refers to one of the following classes: bacteria, fungi, algae, eukaryote, archaea, protozoa and viruses. Suitable microorganisms refer to any of those well established and those novel microorganisms and variants that emerge from time to time.

As used herein, the term “growth” or “microbial growth” means any measurable change attributable to/or occurring within the life history of an organism. The measurable change can refer to an increase in attributes such as mass, cell divisions (e.g., binary fission events or cell doubling resulting in the production of daughter cells), cell number, cell metabolism products, or any other experimentally observable attribute of a microorganism.

The term “recipe”, as used herein may include any traditional microbiological culture medium that may be known to a person of ordinary skill in the art and can further include any growth (or selective) medium comprising any combination of medium components, whether defined or undefined (complex). Examples of medium components and classes of components include carbon sources, nitrogen sources, amino acids, extracts, salts, metal ions, cofactors, vitamins, dissolved gasses, and the like. Similarly, a “recipe” can include various components that might be added to a medium to influence the growth of a microorganism, such as selective and non-selective antimicrobial agents, modulating agents (i.e., agents that may alter microorganism growth, or enrichment agents (e.g., substance that may be required for auxotrophic microorganisms, such as hemin, or substances that may be required by fastidious organisms) or other components that may encourage microorganism growth.

In various embodiments, the term “growth conditions” of the microbe refers to one or more conditions suitable for growth. For example, a “condition” can include one or more parameters required for/or beneficial to microorganism growth. A “condition” can also include other environmental parameters separate from the composition of a culture medium, such as light, pressure, temperature, aerobic/anaerobic and the like. Similarly, a condition can include any of a variety of other parameters that might occur or be imposed, such as: a host organism defensive material or cell (e.g., human defensin proteins, complement, antibody, macrophage cell, etc.), a surface adherent material (i.e., surfaces intended to permit growth, etc.), a physiological, metabolic, or gene expression modulating agent, a physiological salt, metabolite, or metabolic waste material (such as may be produced by living microorganisms or used to simulate late-stage culture growth conditions (i.e., stationary phase conditions), a reduction in nutrient media (simulating, for example, stationary phase conditions). Furthermore, a “condition” may be static (e.g. a fixed concentration or temperature) or dynamic (e.g. time-varying, to simulate pharmacokinetic behavior of intermittent infusions; or to simulate any endogenous or exogenous process affecting microbe response). These definitions of “condition” are intended to be illustrative, rather than exhaustive, and, as used herein, a “condition” can include any endogenous or exogenous parameter that may influence a microorganism.

The term “sequence” refers to the order and composition of biomolecules DNA, RNA, protein or metabolite, in an organism, that can function to identify or aid in identifying or characterizing the organism.

The term “close genetic relative” in certain embodiments of this invention refers to microbes selected from the same genus as the user-desired microbe having at least 90% sequence identity to the user-desired microbe. Here, said “sequence identity” or “identity” in the context of two nucleotide or polypeptide sequences makes reference to a specified percentage of residues in the two sequences that are identical when aligned for maximum correspondence over a specified comparison window, as measured by sequence comparison algorithms or by visual inspection. Suitably, a specified comparison window, is selected from a sequence encoding or representing at least 20, at least 25, at least 30, at least 40, at least 50, at least 75, at least 80, at least 85, at least 90, at least 95, at least 100, at least 105, at least 110, at least 115, at least 120, at least 250 or most preferably all of the amino acids of a specified polypeptide being aligned. In certain embodiments, the specified comparison window is all of the residues of the sequences. When percentage of sequence identity is used in reference to proteins, it will be understood by those of skill in the art that residue positions which are not identical, often differ by conservative amino acid substitutions, i.e. wherein amino acids are substituted with amino acids which have similar chemical properties to those amino acids which are replaced. The percent sequence identity may be adjusted upwards to correct for the conservative nature of a substitution. Furthermore, “close genetic relative” can include microbes from the same genus as the user-desired microbe having at least one pathway in common with the user-desired microbe, indicating a growth condition or requirement (e.g. sulfur oxidation pathway, requiring sulfur for growth).

The term “16s metagenomic sequencing” as used herein refers to sequencing the 16S ribosomal RNA (or 16S rRNA) gene using methods commonly understood by one of skill in the art. 16S rRNA is a component of the 30S small subunit of prokaryotic ribosomes. Sequence information generated from the 16s rRNA gene, is used for species identification of microbes in a sample.

### BRIEF DESCRIPTION OF DRAWINGS

FIG. 1 depicts a schematic of an exemplary workflow of the present disclosure. The “unknown sample” without limitation, may refer to soil, water, aerosol, permafrost, clinical specimen, etc. The analysis workflow as indicated, comprises evaluating input sequences to identify and characterize the microbe and utilizing GrowSEQ recipe database to match the “best guess” recipe for a known microbe. If an unknown microorganism is detected from the input sequence, predictive AI tools or relatedness to closest species can be used to generate novel growth recipes for the user. The invention can also be characterized by any of the descriptions in the FIGURE.

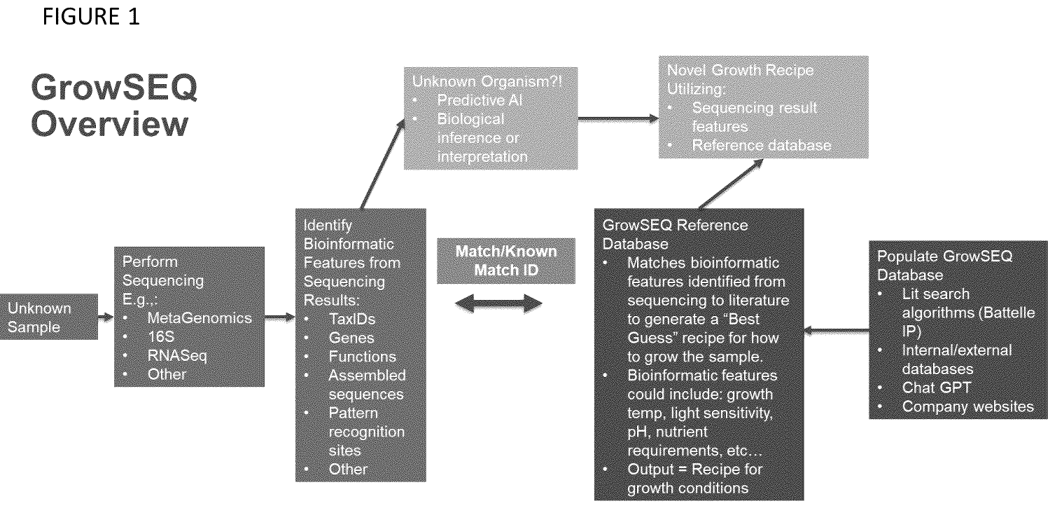

### DETAILED DESCRIPTION OF THE INVENTION

In some embodiments, GrowSEQ may use a database of recipes indexed based at least on biological and physical aspects of microbes. For example, aspects such as DNA or RNA characteristics, particular or relevant genes that indicate specific growth requirements, and morphology aspects of the microbe, among others. Recipes vary greatly in the same wide variety as microbes. For example, every species of bacteria has optimal growing conditions with a wide range of possible chemical compositions, and differences in the media used to grow them. Agar and liquid suspensions are but two possible media hosts. A recipe will include at least any aspect that may possibly affect the growth of a microbe during its life cycle. A recipe may also include exemplary aspects such as, for example, additional additives, correlations if needed with possibly different stages of growth, or recommendations for scaling based on densities of microbes, optimal lighting, aerobic or anaerobic conditions and other environment factors including whether specific biosafety levels may be recommended for pathogenic microbes.

The GrowSEQ database of recipes may organize and index any relevant information without limitation. Sources of information may include, but not limited to, technical literature and the input of subject matter experts during the course of research or analysis of microbes. The database may be updated at any time as new information becomes available by any means known in the art. In some embodiments, a user interface may be provided, to allow record management functions that may include creating, deleting, editing, indexing or otherwise modifying the recipe database. In other embodiments, the GrowSEQ database may be linked to one or more remote databases containing information and may synchronize its contents with the remote databases. GrowSEQ also may curate or edit any synchronized contents.

Embodiments described herein may analyze data from any biological specimen that contains DNA/RNA, for example bacteria, archaea, fungi, virus, or eukaryote. Since GrowSEQ utilizes a bioinformatic approach to search through either 16s metagenomic sequencing, transcriptome data, amplicon sequencing data, or full genome sequencing data, any sample may be used if the nucleic acid (DNA and/or RNA) remains intact.

Alternatively, GrowSEQ may use a bioinformatics approach to analyze proteomic sequencing generated via mass spectrometry or metabolomics data to identify organisms, by querying publicly and privately available proteomics or metabolomics databases. In another embodiment, if proteins are found, the corresponding nucleic acids sequence could then be determined via tBLASTn search available from NCBI BLAST and evaluation via UniProt protein database.

The GrowSEQ process may begin with sequencing, usually next generation sequencing as it may be known in the art. The methodology of extraction of nucleic acids from biological samples and subsequent sequencing may be performed using standard protocols well known in the art. For example, a biological sample of interest is collected. A “sample” may be any material or substance that appears to contain nucleic acids indicative of a living organism, such as virus, bacteria, eukaryote, or archaea without limitation. The samples may be processed via nucleic acid extraction, again, using methods and systems well known in the art. Then, next generation sequencing may be used to evaluate the 16s rRNA, targeted amplicon regions and/or full genomes if available.

The sequencing and identification aspects are integrated with recipe selection, as is described below in the following steps.
Step 1 of GrowSEQ is to process sequencing data and evaluate against publicly available databases. In one embodiment, GrowSEQ utilizes a Python script that implements tools such as NCBI BLAST or other software tools to assist with metagenomic analysis. The result of the analysis becomes input to the next step of GrowSEQ on an integrated basis.
In Step 2, GrowSEQ utilizes the metagenomic analysis of the sample to provide a list of microbes obtained from the sample. The list of microbes is then cross-referenced to a GrowSEQ recipe database.
In Step 3, one or more recipes that will permit growth may be shown to a user, via a user interface, for a desired microbe or for all microbes on the list in parallel.
In Step 4, GrowSEQ provides a recommendation that will permit growth of the microbe of interest. In an aspect, the recommendation may include options ranked or sorted according to a pre-specified criterion, for example, speed of growth, cost, microbe concentration, environment conditions or available tools within the laboratory, without limitation.
In an embodiment, if a microbe on the list does not have a known recipe in a GrowSEQ recipe database, then GrowSEQ may conduct a BLAST search with the microbe DNA/RNA/peptide sequence. Using BLAST search results, or similar tools or methods, GrowSEQ may identify the next closest relative and provide a recipe for the growth of the next closest relative. It will be appreciated by one of ordinary skill in the art that the next closest relative may serve as an optimal starting point for growing a previously unknown species.
Particularly, in an aspect, GrowSEQ correlates species that are close evolutionary relatives based on a combination of input such as DNA/RNA information, proteome or pathway analysis methods. In the following non-limiting example, species A and species B are close evolutionary relatives. Because close relatives will often grow well under the same conditions and subject to the same recipe, the GrowSEQ tool will return a recipe for Species A when Species B is under test and there is no recipe for species B.
In the event that a recipe for a particular species does not exist, or when the user-desired microbe is unknown, GrowSEQ will utilize amplicon sequencing or whole genome sequencing data if available, and search for key genes involved in specific processes or pathways for microbial growth. For example, sulfur-oxidizing bacteria may contain specific genes needed for utilizing thiosulfate and sulfide as sources of energy for growth. If GrowSEQ identifies these growth-oriented genes within the DNA/RNA/proteome of an unknown species as supplied via the biologic sample described above, GrowSEQ may improve its ability to suggest a potentially successful recipe through the addition of the indicated and/or specified ingredients.
In Step 5, the user may select one of the suggested recipes. The user's selection may depend on the context of the user's research; for example, the recipe for growing microbes in significant numbers may be different to the recipe for growing small concentrations. Upon selection, one or more recipes will be provided in a list format along with materials and steps required to prepare the appropriate media. As described above, the recipe may include any data that is relevant and useful. In an aspect, the apparatus that is appropriate for housing a culture may be specified. Non-limiting examples may include flasks for liquid, plates for agar, anaerobic or aerobic incubator and the like.
In Step 6, the user has the option to input optical density and other growth metrics to evaluate the success of the growth recipe generated by GrowSEQ. The inputs may be made by any means known in the art, such as a user interface.

GrowSEQ will accept inputs in order to optimize its recipe repository and improve the quality and accuracy of its recipes. For example, in a non-limiting aspect, GrowSEQ may leverage machine learning and/or artificial intelligence aspects to learn through iterative training, using techniques such as challenge steps, or various kinds of neural networks such as for example convolutional neural networks or recurrent neural networks. Accordingly, GrowSEQ may supply increasingly accurate recipe suggestions for future unknown microbes over time.

There are many examples where use of GrowSEQ may save lives or significantly prevent harm. In one example, the arctic permafrost contains hosts of organisms and many microbes that have never been characterized. Some of these could contain harmful human pathogens. To evaluate the microbes that are unknown, researchers will need to grow them successfully in a laboratory setting. The system and method within GrowSEQ may generate recipes for this growth and expedite understanding of the new microbes. A second example is directed to beneficial microbes. The many regions in the world that have become landfill (or ocean bearing) are reservoirs for plastic waste. Amongst the various microbial populations that are found in these areas, some microbes can degrade plastic waste. Traditional research methods require a subject matter expert or person with ordinary skill in the art to conduct a literature search and/or leverage personal experience on how to grow the uniquely beneficial microbes. Alternatively, if there exists no information about the unknown microbe in the literature, the challenges of trial and error can significantly delay or impede successful attempts at growth and maintenance of the microbes. By using GrowSEQ which can generate “best-guess” recipes from various types of sequence input, the researcher can more rapidly select for and grow the microbe(s) of interest, thereby increasing the chance of success at propagating these microbes, lowering costs by reducing the number of troubleshooting attempts and saving time.

The embodiments described herein may find applicability in any computing or processing environment. Various embodiments may be implemented in hardware, software, or a combination of hardware and software. Embodiments may be implemented using one or more computer programs on programmable computers and/or on a computing or communication device connected to a network, that each includes a processor, and a storage medium (e.g., a remote storage server) readable by the processor (including volatile and non-volatile memory and/or storage elements) without limitation.

Any software programs used in any embodiments may be implemented in a high-level procedural or object-oriented programming language to communicate with a computer system and/or user devices. Also, the programs can be implemented in assembly or machine language. The language may be a compiled, interpreted, or scripted language without limitation. Computer programs may be stored on any suitable storage medium or device (e.g., CD-ROM, hard disk, or magnetic diskette, optical or other drive) that is readable by a general or special purpose programmable machine for configuring and operating the computer when the storage medium or device is read by the computer to execute software instructions. Computer resources, such as processing and storage, may be configured in a single device or spread among several devices in the same location or distributed in remote locations, interconnected by a suitable network, as is well known in the art, without limitation.

Databases used in various embodiments may be any type of information storage and access system commercially available, publicly available or built for the purposes as described in this disclosure. Databases may be stored on a single server or distributed in a cloud-operating environment. Databases may be locally stored or connected via a network to user devices, which may include any form of computing device including mobile devices.

The databases used in various embodiments may be accessed by a variety of user interfaces either commercially available or built for the purposes described in this disclosure. The user interfaces may be built for use on any computing device as is known in the art, together with security measures to assure only authorized users may search for, view, edit or create data records.

The embodiments described herein may also use machine intelligence without limitation, specifically to include aspects of artificial intelligence, particularly for the correlation functions between candidate microbes (microbes yet to be characterized with a recipe) and available recipes together with reports on extent of success of the recipe in fostering growth. However, machine intelligence may be used for any aspect of this disclosure.

It will be appreciated that an exemplary computing system is merely illustrative of a computing environment in which the herein described systems and methods may operate, and therefore does not limit the implementation of the described systems and methods in possibly different computing environments that may have different components and configurations. In other words, the inventive concepts described herein may be implemented in various computing environments using various components and configurations. Moreover, those of skill in the art will appreciate that the herein described apparatuses, engines, devices, systems and methods are susceptible to various modifications and alternative constructions. There is no intention to limit the scope of the invention to the specific constructions described herein. Also, it should be apparent that the embodiments disclosed herein are not limited to a specific architecture or programming language. Rather, the systems and methods described herein are intended to cover all modifications, alternative constructions, and equivalents falling within the scope and spirit of the disclosure, any appended claims, and any equivalents thereto.

While several inventive embodiments have been described and illustrated herein, those of ordinary skill in the art will readily envision a variety of other means for performing the function or obtaining the results and advantages described herein, and each of such variations and/or modifications is deemed to be within the scope of the inventive embodiments described herein.

Example: Identifying growth conditions for user-desired microbial species. Input sequence is generated in the following steps, using methods and systems known to persons with ordinary skill in the art, prior to analysis by GrowSEQ.
1. A sample of interest is collected via manual or automated means (any material that contains nucleic acids indicative of living organisms; eg. Virus, bacteria, eukaryote, archaea, fungi, etc)
2. Samples are processed for nucleic acid extraction.
3. The sample nucleic acids undergo library preparation depending on downstream sequencing approach. For example, qPCR and/or next generation sequencing is utilized to evaluate the 16s rRNA, targeted amplicon regions, and/or full genomes.

GrowSEQ steps:
1. Sequencing data is processed and evaluated against publicly available databases for species level identification. This can be conducted via a python script that uses tools such as NCBI BLAST or other existing software for metagenomic analysis.
2. A list of species from the sample is provided and cross-referenced to a recipe database created for the GrowSEQ tool through literature searches and SME input.
3. GrowSEQ suggests top recipe that will permit growth of the species of interest or group of species.
   a. If species does not have a known recipe in the data repository, then GrowSEQ will conduct a BLAST search with the unknown species' DNA/RNA, identify the next closest relative, and provide the recipe for that species. This would serve as the best starting point for growing an unknown species.
   b. Alternatively, for these unknown species, if amplicon sequencing or whole genome sequencing data is made available, GrowSEQ will also search for key genes involved specific processes for growth. (e.g. Sulfur-oxidizing bacteria can contain specific genes needed for utilizing thiosulfate and sulfide as sources of energy for growth. If GrowSEQ identifies these genes in an unknown species, it can better suggest a potentially successful recipe through addition of those ingredients.)
4. User has opportunity to select the suggested recipe or make an alternative choice.
5. Upon selection, recipe will be provided in a list format along with materials and steps required to make the media, apparatus needed for housing the culture (e.g. flasks for liquid, plates for agar, anaerobic for aerobic incubator etc)
6. User can use GrowSEQ to input OD and other growth metrics to evaluate success of growth.
7. GrowSEQ will take said inputs and leverage ML/AI algorithms to learn through iterative training and challenge steps to provide increasingly accurate recipe suggestions for future unknown species.

What is claimed is:

1. A method for microbial growth, comprising:
a. receiving and analyzing of sequencing data from one or more microbes supplied in a biological sample, wherein sequencing data may include 16s metagenomic sequencing data, transcriptome data, amplicon sequencing data, full genome sequencing data, proteomic sequencing data, metabolomics data and pathway analysis;
b. identifying of microbial species in the sample;
c. accessing a repository of growth recipes and recommending at least one growth recipe for each microbe on the list of microbes;
d. in the event that the microbial recipe for a species-type does not exist, generating a recipe that matches a close genetic relative; and
e. generating a recipe for a particular microbe by integrating information from a combination of input sequencing data as stated in step (a).

2. The method of claim 1 further comprising expanding the repository of growth recipes using laboratory tested user feedback.

3. The method of claim 1 wherein the species comprise known and unknown species.

4. The method of claim 1, further comprising generating microbial growth conditions uniquely suited to the concentration of the microbial sample.

5. The method of claim 1, comprising generating microbial growth conditions in a batch of a first amount and generating microbial growth conditions in a batch of a second amount wherein the second amount is at least ten times larger than the first amount and wherein the growth conditions of the second amount differ from those of the first amount by more than merely multiplying all factors.

6. The method of claim 2, wherein the repository of growth recipes and growth conditions increases in number and accuracy from user feedback.

7. The method of claim 1, wherein the microbial sample contains one or more unknown microbial species.

8. The method of claim 7, wherein the microbial sample contains more than one unknown microbial species.

9. The method of claim 7 wherein the microbial sample comprises at least one known microbial species.

10. The method of claim 1 wherein the sequencing data comprises transcriptome data, proteomic sequencing data, and full genome sequencing data.

11. The method of claim 1 wherein the sequencing data includes mixed population shotgun metagenomic sequencing.

12. The method of claim 1 wherein the sequencing data comprises 16s metagenomic sequencing data.

13. The method of claim 1 wherein the sequencing data comprises pathway analysis and wherein the pathway analysis comprises KEGG, Gene Ontology, STRINGDB, PANTHER, MSigDb, Pathway Commons, NCI PID.

14. The method of claim 1 wherein the microbes comprise a virus.
